(19) United States
(12) Patent Application Publication
(10) Pub. No.: US 2023/0190652 A1
(43) Pub. Date: Jun. 22, 2023

(54) COMPOSITION FOR THE TREATMENT OF COVID-19 AND TREATMENT METHOD

(71) Applicant: DERMOPARTNERS,S.L., Rafelbunyol (ES)

(72) Inventor: Gabriel Serrano Sanmiguel, Rafelbunyol (ES)

(21) Appl. No.: 16/918,260

(22) PCT Filed: Apr. 16, 2020
(86) PCT No.: PCT/ES2020/070245

§ 371 Date: Jul. 1, 2020

(51) Int. Cl. A61K 9/127; A61K 9/00; A61K 38/40; A61K 33/30; A61K 31/375; A61P 31/14

(52) U.S. Cl. CPC A61K 9/127; A61K 9/0053; A61K 38/40; A61K 33/30; A61K 31/375; A61P 31/14

(57) ABSTRACT

New formulation to be used in the treatment and prevention of infections caused by COVID-19 and other coronaviruses, consisting of a food supplement the main active ingredient of which is liposome-encapsulated lactoferrin. As secondary active ingredients, it includes zinc salt and vitamin C, also encapsulted in liposomes. This composition is intended to be used as a treatment for mild or acute cases of COVID-19, and to prevention infection by those who are not infected.

New composition to be used in the treatment and prevention of infections caused by COVID-19 and other coronaviruses.

### FIELD OF THE TECHNIQUE

The aim of this invention is its application in the field of the medical and pharmaceutical industry, since it is a composition used to prevent and/or treat viral infections, specifically caused by coronaviruses, which have recently led to a worldwide pandemic due to the absence of vaccines and/or effective treatments against this virus.

### STATUS OF THE TECHNIQUE

There are numerous pharmaceutical formulations that include lactoferrin as its main active ingredient. Several are already patented, but most are dermatological treatments for diabetes, such as patents EP2323625, EP2692355, EP3603621, EP3210618, and US2016206707 among others.

However, there are no references regarding the use of pharmaecutical formulations including liposome-encapsulated lactoferrin for the treatment and prevention of infections caused by COVID-19 or other coronaviruses.

### DESCRIPTION OF THE INVENTION

Based on the status of the previous technique, the aim of this invention is to provide a pharmaceutical formulation that can fight and/or prevent infection caused by coronavirus. The composition of this invention was obtained through the encapsulation of its component in liposomes and was tested with a clinical trial.

The president and founder of the Sesderma laboratories, inventor of this patent, knows about the multiple effects of lactoferrin on the human body. Their product Lactyferrin™ Forte Drinkable, which contains liposomal lactoferrin, has been on the market for 14 years. The possible effectiveness of this product against COVID-19 was considered, for which a clinical trial was carried out.

A prospective observational study was carried out on 75 patients with symptoms typical of COVID-19, who tested positive to IgM/IgG rapid test. Patients were isolated and treated at home using remote systems, reviewed twice a day for 10 days, and monitored for up to 1 month. A liposomal bovine lactoferrin (LLF) nutritional syrup food supplement (32 mg of LF/10 ml plus 12 mg of vitamin C) was administered orally in 4 to 6 doses per day for 10 days. In addition, a zinc solution was administered at the dose of 10 mg/10 ml twice or three times a day. A control group of 12 patients who took only LLF was included. All family members in contact with patients (256 persons) were also treated with half of this dose. The treatment allowed a complete and fast recovery in all patients (100%) within the first 4-5 days. Same treatment at a lower dose prevented the disease in healthy persons directly related with the affected patients. Weakness (95%) followed by a dysfunction in the perception of smell and taste (88%), cough (83%), and muscular pain (67%) were the most frequent complaints.

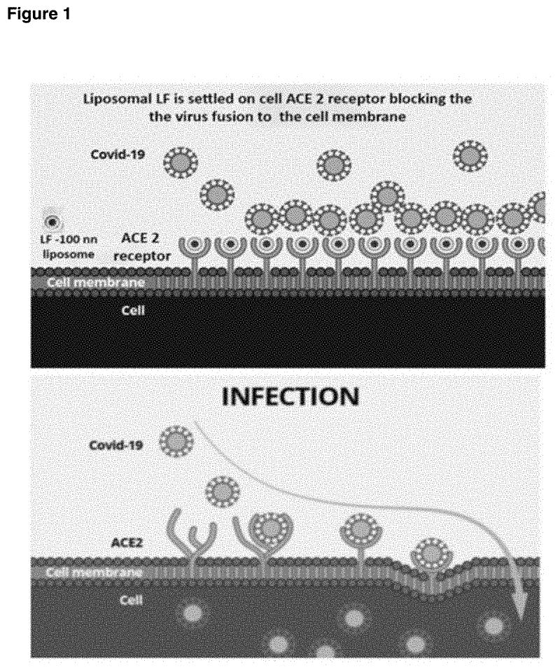

In December 2019, coronavirus disease 2019 (COVID-19) emerged in Wuhan City and rapidly spread throughout China and to all five continents within a few months, creating a pandemic. Since then, search has been carried out for a safe and effective treatment. Clinically, the illness starts with fever, headache, dry cough, fatigue, myalgia, dyspnea, abdominal pain, diarrhea, nausea and vomiting. Disease progression may gradually lead to respiratory failure due to alveolar damage and even death. Laboratory predictors of adverse clinical outcomes include lymphopenia (35-75% of cases), increased values of CRP (75-93% of cases), LDH (27-92% of cases), ESR (up to 85% of cases) and D-dimer (36-43% of cases), as well as low concentrations of serum albumin (50-98% of cases) and haemoglobin (41-50%). Many laboratory abnormalities were instead predictive of adverse outcomes, including increased white blood cell count, increased neutrophil count, decreased lymphocyte count, decreased albumin, increased LDH, ALT, AST, bilirubin, creatinine, cardiac troponins, D-dimer, prothrombin time, procalcitonin and CRP values. Antivirals and hydroxychloroquine were initially suggested as therapies for COVID-19-associated pneumonia in multicentre clinical trials conducted in China. Angiotensin Converting Enzyme 2 (ACE2) is the main receptor for COVID-19 (FIG. 1) and plays a vital role in the entry of the virus into the cell to induce lung infection.

These receptors are highly expressed in the nose, mouth, epithelial respiratory tract, alveolar epithelial cells of the lungs, enterocytes of the small intestine and the brush border of the proximal tubules of the kidney. The localization of ACE2 receptors is associated with tissue tropism and pathogenesis of the viral infection. The disease may cause an upper respiratory tract infection (sinuses, nose, and throat) but most frequently causes a lower respiratory tract infection (trachea and lungs). The COVID-19 infection spreads in the same way as other coronaviruses: mainly through person-to-person contact. Infections range from mild to serious.

In the year 2011, Lang et al. stated that lactoferrin could be potentially useful for the treatment of SARS disease. However, until now, there have been no reports of the clinical use of lactoferrin in patients affected by SARS-COv or COVID-19 infection.

The COVID 19 is an enveloped, RNA virus with a genome of about 30000 nucleotides in length and encodes a nonstructural replicase complex and structural proteins including spike (S), envelope (E), and S2, membrane (M) and nucleocapsid (N) proteins. The spike protein is composed of two units: S1, which mediates the virus binding to receptors on target cells, and S2, which triggers virus and host cell membrane fusion. Angiotensin-converting enzyme 2 (ACE2) is a metallopeptidase: a functional receptor of the virus and is responsible for binding to S protein (FIG. 1) and mediating the virus entry into the target cells. A segment with amino acids 318-510 of the protein S1 is the receptor binding domain for the ACE2 receptor. ACE2 is highly expressed on human lung alveolar epithelial cells, enterocytes of the small intestine and the brush border of the proximal tubular cells of the kidney.

The localization of ACE2 expression is consistent with tissue tropism and pathogenesis of SARS-Co infection. Some patients with COVID 19 infection develop an intense host immune response against the virus. The innate immune response plays an important role in decreasing the viral infection. Many genes may be involved in the innate immune response, such as that encoding lactoferrin, S100A9 and lipocalin 2, which participate in SARS-CoV clearance.

It was observed that LF gene expression was upregulated and that LF was highly elevated (150 folds) in SARS patients in comparison with healthy volunteers. In our experience, CSGF was also upregulated but instead GMCSF was downregulated (unpublished data) after healthy volunteers ingested liposomal LF. It is believed that LF in this infection may work by stimulating NK cell activity and neutrophil aggregation and adhesion.

Lactoferrin is a multifunctional glycoprotein present in several body fluids including breast milk, saliva, tears, semen, vaginal fluid, nasal and bronchial secretions, gastrointestinal fluids, and urine mucosal secretions, and is also present in the neutrophilic granules of leukocytes.

Lactoferrin has a strong antiviral activity against RNA and DNA virus including human immunodeficiency virus, zika virus, Chikungunya, hepatitis C, Sindbis virus, cytomegalovirus, herpes simplex virus, human papillomavirus, and rotavirus. These viruses utilize common molecules such as heparan sulphate proteoglycans (HSPG) on the cell membrane to facilitate the invasion of the cells. These molecules provide the first anchoring sites on the cell surface and help the virus make primary contact with host cells.

LF may be able of preventing the internalization of some viruses after binding to HSPGs, which is present on most cells. This property of LF confers protection to the host against viral infections. LF has an important protective role in the host immune defense against COVID 19 invasion.

Seventy-five patients affected by COVID-19 showing the typical symptoms of the disease and testing positive for SARS-CoV-2 were selected for inclusion in the study. Patients were isolated and treated at home using remote systems, assessed daily for 10 days, and followed up for up to one month. Diagnosis was confirmed by SARS-CoV-2 IgM/IgG antibody rapid test done in whole blood (SARS-CoV-2 IgM/IgG antibody rapid test (Liming Bio, Jiangsu, P. R. China). LLF (Lactyferrin™ Forte drinkable, Sesderma Laboratories, Valencia, Spain) as well as a liposomal zinc (LZ) syrup (Zinc Defense syrup, Sesderma Laboratories, Valencia, Spain) were administered orally in 4 to 6 doses per day. LZ solution was administered at the dose of 10 mg/10 ml twice or three times a day. A control group of 12 patients also received only LLF. The LLF total daily dose ranged between 256 and 384 mg/day. All family members who had contact with the patients (256 persons) were also treated with half of this dose. Patients presenting headache, dry cough and nasal congestions were also treated with LLnasal drops and mouth spray (Lactyferrin nasal drops and Lactyferrin mouth spray) applied 4 times a day. LL in aerosol (SES Nanomist,

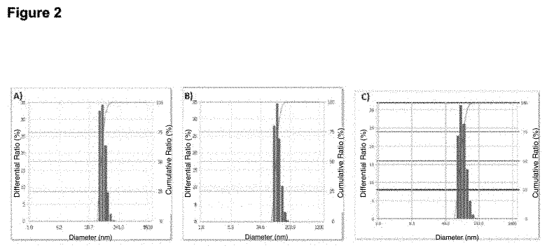

Sesderma Laboratories; FIG. 2) was also administered to all patients experiencing breathing difficulties. All the participants were assessed remotely by the medical team. Patients were monitored daily (at least twice a day) for 10 days and then after 30 days. For every patient and at every session symptoms were scored on a scale of 0 to 3, as follows: 0 absence of symptoms; 1 mild symptoms; 2 moderate symptoms; and 3 severe symptoms. Taste and smell were evaluated on a scale of 0 to 5, where 0 represented the absence of taste/smell (ageusia/anosmia) and 5 non-affected taste/smell.

For the group with the combination treatment (LLF+LZ), the median age of the patients was 42 years; 45% were female. We also included a separated group of four patients who underwent mechanical ventilation and were treated in the hospital. All were alive at the time of follow up. The most commonly reported symptoms were weakness/tiredness (94.44%), loss of smell (83.33%) and taste (88.89%), muscular pain (66.67%) dry cough (61.11%), headache (55.56%), diarrhea (44.4%), runny nose (33.33%), breathing difficulties (27.78%), nasal congestion (22.22%) and odynophagia (22.22%). Other symptoms include fever (38%), cramps (30%), insomnia (50%), night agitation (30%), nausea and intense stomach pain, flatulence, and sore throat (28%), while one patient complained of abrupt and heavy hair loss (1.3%).

No analytical data (radiography or computed tomography) were collected. Patients treated in the hospital were not included as to date we do not have a complete data set.

DAY 0: Data (symptoms) was collected during the first remote contact with the participants. Treatment had not yet begun (Table 1).

TABLE 1
Absence or presence of symptomatology from patients included
in the study before receiving the treatment (day 0).

| Symptomatology (%) | NO | YES |
|---|---|---|
| Dry cough | 38.89 | 61.11 |
| Productive cough | 77.78 | 22.22 |
| Respiratory distress | 72.22 | 27.78 |
| Muscular pain | 33.33 | 66.67 |
| Rhinorrhea | 66.67 | 33.33 |
| Nasal congestion | 77.78 | 22.22 |
| Taste | 11.11 | 88.89 |
| Smell | 16.67 | 83.33 |
| Odynophagia | 77.78 | 22.22 |
| Tiredness | 5.56 | 94.44 |
| Diarrhea | 55.56 | 44.44 |
| Headache | 44.44 | 55.56 |

The percentages of symptoms observed are shown in Table 2 ordered according to severity. The assessed parameters were already mentioned.

TABLE 2
Degree of involvement of symptoms from patients included
in the study before receiving the treatment (day 0).

| Symptomatology (%) | 0 | 1 | 2 | 3 | 4 | 5 |
|---|---|---|---|---|---|---|
| Dry cough | 38.89 | 22.22 | 33.33 | 5.56 | — | — |
| Productive cough | 77.78 | 16.67 | 5.56 | 0 | — | — |
| Respiratory distress | 72.22 | 27.78 | 0 | 0 | — | — |
| Muscular pain | 33.33 | 16.67 | 27.78 | 22.22 | — | — |
| Rhinorrhea | 66.67 | 22.22 | 11.11 | 0 | — | — |
| Nasal congestion | 77.78 | 11.11 | 11.11 | 0 | — | — |
| Taste | 72.22 | 11.11 | 0 | 5.56 | 0 | 11.11 |
| Smell | 72.22 | 11.11 | 0 | 0 | 0 | 16.67 |
| Odynophagia | 77.78 | 22.22 | 0 | 0 | — | — |
| Tiredness | 5.56 | 11.11 | 50 | 33.33 | — | — |
| Diarrhea | 55.56 | 33.33 | 11.11 | 0 | — | — |
| Headache | 44.44 | 27.78 | 5.56 | 22.22 | — | — |

48 hours: Data was collected 48 hours after the first contact with the participants. Patients had already started treatment and some improvements could be observed (Table 3).

TABLE 3
Absence or presence of symptomatology from
patients after 48 hours of treatment.

| Symptomatology (%) | NO | YES |
|---|---|---|
| Dry cough | 50 | 50 |
| Productive cough | 88.89 | 11.11 |
| Respiratory distress | 100 | 0 |
| Muscular pain | 55.56 | 44.44 |
| Rhinorrhea | 83.33 | 16.67 |
| Nasal congestion | 94.44 | 5.56 |
| Taste | 11.11 | 88.89 |
| Smell | 16.67 | 83.33 |
| Odynophagia | 88.89 | 11.11 |
| Tiredness | 33.33 | 66.67 |
| Diarrhea | 88.89 | 11.11 |
| Headache | 100 | 0 |

After the first 48 hours of treatment, headache symptoms disappeared in 100% of patients. The percentage of patients with a dry cough decreased from 61.11% to 50%. Patients reported that they were experiencing significant relief of these symptoms and associated this fact with the application of the nasal drops and mouth spray. The percentage of patients experiencing muscle pain decreased from 66.67% to 44.44%, while the percentage of patients experiencing tiredness/weakness decreased from 94.44% at the start of the study to 66.67% after 48 hours.

For all the patients showing moderate to severe dyspnea, LF nebulization was performed using a Nanomist nebulizer (SES Nanomist, Sesderma laboratories). No significant improvement in taste or smell was reported after 48 hours (Table 3). Improvements in taste and smell were slower compared with the rest of the symptoms. Table 4 (% severity scale for the symptoms) showed that 72.22% of the patients who had a total loss of taste and smell (ageusia/anosmia) at day 0 (Table 2), presented a reduction at 48 hours (44.44% and 38.89% for taste and

TABLE 4
Degree of involvement of symptoms from
patients after 48 hours of treatment.

| Symptomatology (%) | 0 | 1 | 2 | 3 | 4 | 5 |
|---|---|---|---|---|---|---|
| Dry cough | 50 | 33.33 | 16.67 | 0 | — | — |
| Productive cough | 88.89 | 11.11 | 0 | 0 | — | — |
| Respiratory distress | 100 | 0 | 0 | 0 | — | — |
| Muscular pain | 55.56 | 38.89 | 5.56 | 0 | — | — |
| Rhinorrhea | 83.33 | 16.67 | 0 | 0 | — | — |
| Nasal congestion | 94.44 | 0 | 5.56 | 0 | — | — |
| Taste | 44.44 | 22.22 | 11.11 | 11.11 | 0 | 11.11 |
| Smell | 38.89 | 22.22 | 22.22 | 0 | 0 | 16.67 |
| Odynophagia | 88.89 | 11.11 | 0 | 0 | — | — |
| Tiredness | 33.33 | 44.44 | 16.67 | 5.56 | — | — |
| Diarrhea | 88.89 | 5.56 | 5.56 | 0 | — | — |
| Headache | 100 | 0 | 0 | 0 | — | — |

Day 5. All the participants continued to improve and followed the 10-day treatment schedule. At day 5, the percentage of patients with a dry cough decreased from 61.11% at the start of the study to 38.89%. The percentage of patients experiencing muscle pain was reduced from 66.67% to 22.22% while the percentage of those reporting tiredness/weakness decreased from 94.44% to 27.78%. As was observed at 48 hours, 100% of the patients reported having no headache on day 5. No significant improvement in taste and smell was reported (Table 5), as observed with the rest of symptoms. The recovery of the smell and taste of patients was slower.

TABLE 5
Absence or presence of symptomatology from
patients after 5 days of treatment.

| Symptomatology (%) | NO | YES |
|---|---|---|
| Dry cough | 61.11 | 38.89 |
| Productive cough | 94.44 | 5.56 |
| Respiratory distress | 100 | 0 |
| Muscular pain | 77.78 | 22.22 |
| Rhinorrhea | 88.89 | 11.11 |
| Nasal congestion | 88.89 | 11.11 |
| Taste | 11.11 | 88.89 |
| Smell | 16.67 | 83.33 |
| Odynophagia | 88.89 | 11.11 |
| Tiredness | 72.22 | 27.78 |
| Diarrhea | 94.44 | 5.56 |
| Headache | 100 | 0 |

However, a progressive improvement in symptoms was observed (Table 6; % grade of severity of symptoms) and from about 72.22% of patients with total loss of taste and smell (ageusia/anosmia) at day 0, the percentage was reduced to 38.85 at day 5.

TABLE 6
Degree of involvement of symptoms from
patients after 5 days of treatment.

| Symptomatology (%) | 0 | 1 | 2 | 3 | 4 | 5 |
|---|---|---|---|---|---|---|
| Dry cough | 61.11 | 38.89 | 0 | 0 | — | — |
| Productive cough | 94.44 | 5.56 | 0 | 0 | — | — |
| Respiratory distress | 100 | 0 | 0 | 0 | — | — |
| Muscular pain | 77.78 | 22.22 | 0 | 0 | — | — |
| Rhinorrhea | 88.89 | 11.11 | 0 | 0 | — | — |
| Nasal congestion | 88.89 | 11.11 | 0 | 0 | — | — |
| Taste | 38.89 | 11.11 | 16.67 | 11.11 | 11.11 | 11.11 |
| Smell | 38.89 | 5.56 | 22.22 | 11.11 | 5.56 | 16.67 |
| Odynophagia | 88.89 | 11.11 | 0 | 0 | — | — |
| Tiredness | 72.22 | 22.22 | 5.56 | 0 | — | — |
| Diarrhea | 94.44 | 5.56 | 0 | 0 | — | — |
| Headache | 100 | 0 | 0 | 0 | — | — |

The progression of the main symptoms of COVID-19 patients at day 0, 48 hours and 120 hours (day 5) is described below.

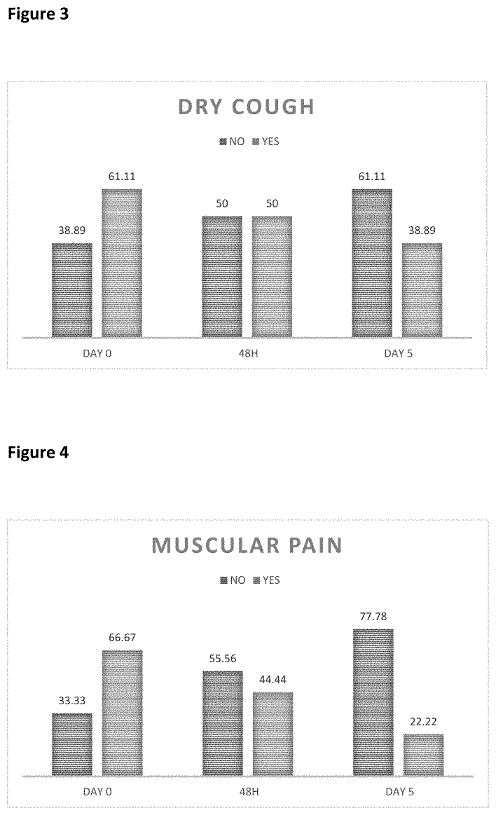

DRY COUGH (FIG. 3): At the start of the study (day 0), 61.11% of the patients had a dry cough. After 48 and 120 hours of treatment, this percentage was reduced to 50% and 38.89% respectively. The treatment notably reduced the incidence of dry cough. BREATHING DIFFICULTY (Table 3): All the patients who reported difficulty breathing at the start of the study (day 0) showed some improvement at 48 hours.

MUSCULAR PAIN (FIG. 4): At the start of the study (day 0), 66.67% of the patients reported muscle pain. However, this percentage was reduced to 44.44% and 2.22% after 48 and 120 hours, respectively. The treatment significantly reduced the strong muscle pain associated with COVID-19.

TIREDNESS (FIG. 5): At day 0, 94.44% of the patients reported feeling tired. After 48 and 120 hours of treatment, this percentage was reduced to 66.67% and 27.78% respectively.

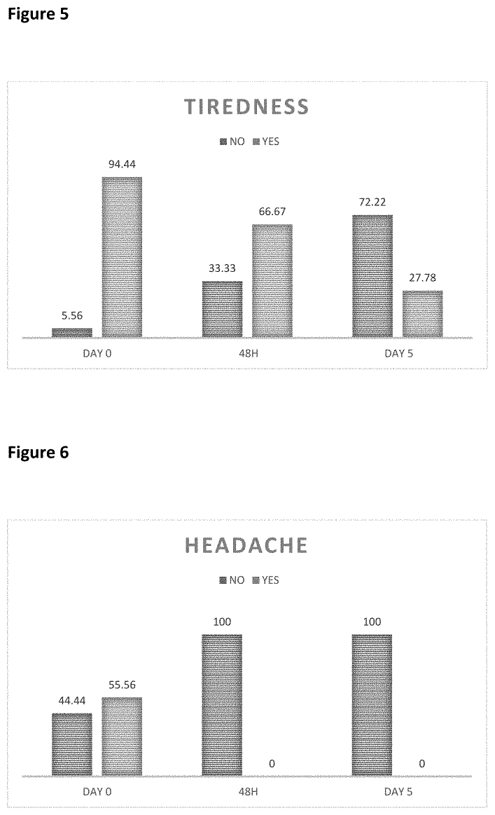

HEADACHE (FIG. 6): At day 0, 55.56% of the patients presented with an intense headache. After 48 and 120 hours none of these patients reported having a headache.

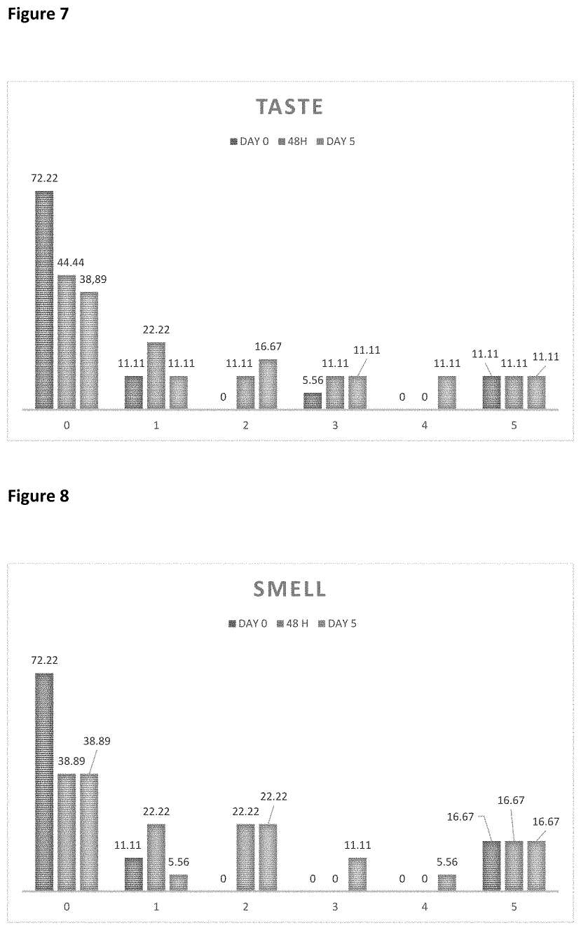

TASTE (FIG. 7): At day 0, 72.22% of the patients had ageusia (total absence of taste), while 11.11% had no taste involvement. The rest of the patients had hypogeusia (partially reduced sense of taste). After 48 hours of treatment, the percentage of patients with ageusia decreased to 44.44% and then further decreased to 38.89% at 120 hours (day 5), implying a partial and progressive return of the sense of taste. At day 10, all the affected patients had completely recovered their sense of smell and taste. Most of the patients with ageusia of not being able to recognize the flavor of food (candy, seafood, chili).

SMELL (FIG. 8): At day 0, 72.22% of the patients had anosmia (total absence of smell), while 16.67% of patients showed no olfactory involvement. The rest of the patients had hyposmia (partial reduced sense of smell). After 48 hours, the percentage of patients with anosmia decreased to 38.89 and remained the same at 120 hours. The sense of smell had completely returned in 95% of the cases at day 10.

Results obtained in the control group taking LLL were very similar.

Analysis of these results indicated that oral treatment with LLF and LLF+LZ allows for fast recovery in 100% of patients within the first 4-5 days. The same treatment but at a lower dose seems to exert a potential preventive effect against COVID-19 in healthy people directly related to the affected patients. Importantly, combination of the oral and topical treatments provided significant relief of the headache and dry cough.

Some of these patients had sinus congestion. Dietary supplementation of LLF an LZ supported and enhanced the immune system response through their antioxidant, antibacterial, and antiviral properties.

The most frequent symptom in our patients was a very heavy sensation of tiredness or weakness (95%) followed by an alteration in the perception of smell and taste (88%), cough (83%), muscular pain (67%), headache (56%) and diarrhea (44%). Most symptoms improved significantly during the first five days: headache (100%), muscular pain (78%), taste (72%), smell (17%), weakness (72%) and dry cough (61%). On day 5, all patients presented no symptoms and complete recovery was observed in all persons (100%). Olfactory and taste disfunction were the most predominant symptom in our cases and occurred very early reflecting the massive entrance of viral particles in the oral and nasopharyngeal mucosa.

These alterations were described in the severe acute respiratory syndrome (SARS). Patients were unable to recognize the taste of foods, even sweet or spicy foods, and to smell (anosmia) fragrances or regular house cleansers (alcohol, kitchen detergents or chlorine bleachers). Angiotensin Converting Enzyme 2 receptor (ACE2), the main receptors for COVID-19, are highly expressed in the nose and mouth, particularly in the base of the tongue and throat sites for which the virus has great tissue tropism.

Nose, mouth and throat topical care with nasal drops, mouth wash, throat sprays and even liposomal based toothpastes could be of help to reduce the viral load. These alterations may be regarded as a transient olfactory and taste dysfunction and requires further follow up and investigation. Patients undergoing chemotherapy may improve taste and smell dysfunction through the intake of LF, improving the oral immunity of cancer patients.

We found that the use treatment of LL nasal drops was very useful to relieve not only the respiratory symptoms but also cough, headache and smell and taste dysfunction. 28% of our patients presented breathing difficulty. In these cases, we recommended the use of the LF liposomal aerosol inhalation device 4 times a day, with great success. The inhalation was well tolerated, and any side effects were reported.

Inhalation through the LLF nebulizer was also recommended to two of the four patients admitted to hospitals. LF is a natural protein present in bronchial secretions and all the content in the liposome is biocompatible and biodegradable.

Aerosol liposomal therapy has been employed for years with good results and a wide safety profile. It has been employed with ribavirin syncytial virus infection and rhinovirus infections. LF is a hydrophilic chemical that behaves as amphiphilic because of its encapsulation in PC liposomes. LF reaches high enough concentrations within the lungs and provides a delayed release of LF in the target organ, the respiratory tract.

LF nanoliposomes have a size of 100 nm, but once it is nebulized through the airways its size is reduced to 50 nm because on the hole of the device there is a filter that reduces the size of the nanosome. The liposome itself may interact with the natural surfactant (phospholipid and proteins) of the target tissue (lungs) and PC may also exert a strong anti-inflammatory effect.

COVID-19 lung infections may alter total pulmonary surfactant and changes its composition, thus decreasing the availability of phospholipids which decreased pulmonary function as it happens in chronic obstructive pulmonary disease. Without surfactant, alveoli tend to collapse during normal tidal breathing, resulting in diminished lung compliance.

Lactoferrin is a globular protein belonging to the family of transferrins and showing great affinity for iron ions. Lactoferrin is a defense protein found in human milk, where it is present at relatively high concentration (1 g/L), especially in the calostral stage (up to 7 g/L). It is also present in other body fluids (tears, semen, vaginal fluid, saliva, bronchial secretions, etc.). It is synthesized by blood neutrophils and various types of cells including some acinar cells. The levels of endogenous lactoferrin increase during infection and inflammation.

LF has antibacterial and antiviral properties, modulates the immune system and protects against intestinal inflammation. Lactoferrin may influence leukocytes by increasing the activity of NK cells, neutrophils and macrophages.

It increases the production of cytokines and nitric oxide and limits the growth of pathogens. Promotes maturation of immune cells, e.g. T and B lymphocytes. It can reduce the symptoms of allergies by blocking the release of histamine from intestinal mast cells. It can probably be helpful in the prevention and treatment of the effects of obesity and may promote reduction of visceral fat. It may help to improve the condition of the skin in ailments such as acne, skin inflammation and psoriasis.

In the present study, we used a liposomal lactoferrin (LLF), registered as a food supplement (Lactyferrin™), and we found this product to be safe and effective for treatment and prevention of COVID-19. 75 patients positive for COVID-19 infection were successfully treated. Most patients responded during the first 3 to 4 days of treatment with Lactyferrin™, although treatment was recommended for 10 days.

Individuals in contact with symptomatic patients were also treated with half of the curative dose, resulting in disease prevention. The combination of liposomal LF and liposomal zinc is very effective and safe for treatment of this potentially fatal disease.

The liposomal bovine LF-based nutritional food supplement, Lactyferrin™ Forte drinkable (Sesderma laboratories, Valencia, Spain) is registered in the European Union (Ireland), as well as by the United States Food and Drug Administration as a food supplement. The European Food Safety Authority recognizes bovine LF as a dietary supplement with medicinal properties, and it is classified as a novel and safe food (‘Generally Recognized as Safe’), with no contraindications.

Total adult doses of non-encapsulated LF range between 1.4 and 3.4 g, and this supplement is frequently used to strengthen the immune system. Lactyferrin contains LF (32 mg/10 ml and vitamin C 12 mg/10 ml). Both substances are hydrophilic and have very limited gastric absorption. The liposome, with its closed phospholipid bilayer vesicular system, could encapsulate both hydrophilic (lactoferrin, vitamin C, zinc) and hydrophobic drugs (vitamin A).

The liposomes we made are based on phosphatidyl choline (PC) which is a biocompatible and biodegradable chemical. LF in its free form is degraded within the stomach by the action of hydrochloric acid and hydrolytic enzymes (proteases, pepsin); hence, the bioavailability of the free form is very limited.

Therefore, LF and vitamin C are encapsulated in a 100 nm lipid bubble or nanoliposome, made from soybean phosphatidylcholine (PC) in Lactyferrin™. The nano-liposome protects LF from destruction by digestive secretions and allows the intact protein to travel through the duodenum and reach the general circulation, from where its bioavailability is very high.

LF encapsulated is in this way is protected from pepsin and hydrolysis by proteases. It is also important to note that free LF is rapidly cleared from the circulation, limiting its therapeutic potential. Therefore, it is necessary to encapsulate it in liposomes to improve plasma stability.

The PC used to make liposomes is an ubiquitous, naturally occurring phospholipid molecule, which is the major lipid in cell membranes and blood proteins. Further, PC serves as the main physiological source of choline, an essential nutrient and precursor to the neurotransmitter, acetylcholine. PC is also necessary to produce surfactants, which are critical for lung function and gastrointestinal health. The terms “phosphatidylcholine” and “lecithin” are sometimes used interchangeably; however, lecithin is a mixture of several lipids and phospholipids. PC is necessary for the composition and repair of cell membranes and vital for normal liver function. Research indicates that PC has a beneficial role in the prevention and treatment of various forms of liver disease and toxicity. PC protects liver cells from viral damage, reduces fibrosis, and prevents cell death from drugs, alcohol, and other chemical toxins.

The protective effects of LF range from anticancer, anti-inflammatory, and immune modulator properties, to antimicrobial, antifungal, and antiviral activities against many microorganisms. This wide range of activities is made possible by action mechanisms involving not only the capacity of LF to bind iron, but also interactions of LF with molecular and cellular components of both hosts and pathogens.

The antibacterial activity of LF is related to deprivation of environmental iron, which is essential for bacterial growth, while its antiviral activity is associated with its role as a competitor of cell membrane receptors commonly used by viruses to enter cells.

Specifically, LF is an ACE2 blocker, and prevents the binding of the virus spike protein S to the host cell, blocking the virus from fusing with the cell membrane. Liposomal LF can also suppress viral replication after entry into the cell, as in the case of HIV. Furthermore, some HIV-1 infected patients show decreased levels of plasma LF and in others, the lack of lactoferrin (and secretory IgA) found in the oral cavities of persons with HIV correlated strongly with the frequent infections in those areas.

Nanoliposomes also have beneficial effects related to their size and composition (PC); they are smaller size (100 nm) than the virus (150 nm), they can compete to reach target cells, where they settle before the virus.

We have also demonstrated that specific doses of liposomal LF can prevent COVID-19. Lower doses do not prevent the infection (unpublished data).

In addition, by using lactoferrin, we have contributed to successful treatment of at least four intubated hospitalized patients, who were high-risk (high ferritin, IL-2, DD2 levels) and had virtually absent vital signs. LF significantly reduces the hyperimmune reaction observed in patients in a critical condition and suffering from an aggressive pro-inflammatory cytokine (IL-2 and 6) storm, normalizing or decreasing IL-6, TNF alpha, ferritin, and DD2 parameters, and protecting the lungs against acute respiratory distress. LF has immunomodulatory and anti-inflammatory properties[32,40], which are important in the pathophysiology of serious infections. Further, LF has a crucial immunomodulatory role in maintaining immune and physiological homeostasis and limiting tissue damage by modulating cytokines, chemokines, and cell surface receptors involved in signaling cascades. The restorative and homeostatic roles of LF are notable in the context of the ‘systemic inflammatory response’, which describes the physiological response to serious insults such as sepsis. The concept of a ‘cytokine storm’, reflects the hyper-induction of inflammatory responses that result from uncontrolled immune activation, and these clearly respond to oral administration of LF.

LF is useful for treatment of the most severe cases of COVID-19 because of its ability to modulate overreactive immune and inflammatory responses to viral infections, as we have observed in at least four patients. LLF might play a role in the disturbances of iron metabolism observed during inflammation and conditions with increased neutrophil turnover. In this event, a lot of released cytokines and/or inflammatory products such as interleukin 1, endotoxin, TNF-alpha and immune complexes may trigger the release of lactoferrin from the neutrophils. Lactoferrin will bind to its membrane receptor in monocytes/macrophages and trapped in this position it might prevent the transfer of iron from the macrophage to serum transferrin. Iron released from senescent phagocytosed red cells might in this way be captured by membrane-bound lactoferrin and transferred back to intracellular ferritin.

We have been using liposomal LF to treat various medical conditions for the past 14 years. We also tested the neuroprotective effects of liposomal LF in a Caenorhabditis elegans model by evaluating both phenotypic and transcriptome responses. The LF-based product protected against acute oxidative stress and extended the lifespan of C. elegans in a dose-dependent manner. Furthermore, paralysis of the transgenic C. elegans strain, CL4176, caused by Aβ1-42 aggregates, was clearly ameliorated by treatment with LF. Transcriptome analysis of treated nematodes indicated that it led to immune system stimulation, together with enhancement of processes involved in the oxidative stress response. The LF-based product also improved processes involved in protein homeostasis, cellular adhesion, and neurogenesis in the nematode. We conclude that LF provides protection against aging and neurodegeneration, modulating processes involved in the oxidative stress response, protein homeostasis, synaptic function, and xenobiotic metabolism. The liposomal LF-based product was also able to stimulate the immune system, as well as improving reproductive status and energy metabolism. Together, all these findings suggest that oral supplementation with liposomal LF could benefit the immune system and improve antioxidant capacity.

Many patients with COVID-19 admitted to our local hospitals are over the age of 70 and have very low zinc levels, which can contribute to the severity of the infection. Zinc is also hydrophilic and exhibits poor absorption through the gastrointestinal tract. Interestingly, the administration of nano encapsulated zinc might support the recovery of patients with COVID-19 infection.

Zinc has also shown a potent antiviral effect. In polio virus experiments, zinc inhibited viral infection when incubated with cells after viral fusion, and the level of inhibition was correlated with the degree of zinc saturation. Zinc supplements have previously been proposed for administration to patients with COVID-19. Zinc may also influence the metalloproteases involved in the process of coronavirus fusion, by decreasing both cell entry and cell-cell fusion.

LF can be used alone or in combination with zinc, and both supplements are non-toxic. They can also be used as adjuvant treatments, alongside conventional antiviral drugs or hydroxychloroquine, as shown for treatment of hepatitis C virus (HCV), where LF decreased the HCV RNA titer by contributing to the effectiveness of therapy with combined interferon and ribavirin.

LF has a great potential for use as an adjunct treatment for patients with viral diseases. From our experience with COVID-19 home-isolated patients, and careful contact tracing, we conclude that liposomal LF can prevent and cure the infection in a dose dependent manner. This treatment is also indicated in patients with severe disease, as we observed in four patients who were critically ill. The doses we recommend for treatment and prevention of COVID-19 are provided in the addendum below. This mentioned treatment is completely free of side effects.

Empirical treatment with hydroxychloroquine plus azithromycin is being promoted by some institutions. However, clinical studies are not completed and hydroxychloroquine is not free of side effects and may induce a wide range of adverse effects. Cardiovascular, dermatologic, gastrointestinal, hematologic, hepatic, hypersensitivity, metabolic, musculoskeletal, nervous system, ocular, psychiatric and respiratory side effects have been described. In particular, cardiac rhythm alterations have been observed in a patient with systemic lupus erythematosus who developed syncopal episodes resulting from significant QT interval prolongation. This was corrected after discontinuation of the drug. The administration of azithromycin is a paradox. Antibiotics don't have any effect on viruses and the drug is administered due to its immune modulatory effect, but unfortunately azithromycin shares with hydroxychloroquine the potential to induce QT-segment prolongation, causing abnormal changes in the electrical activity of the heart that may lead to a potentially fatal irregular heart rhythm.

Antibiotics disturb the intestinal microbiota which has already been destroyed by COVID-19, and probiotics administration is recommended. Elderly patients taking antibiotics may develop intestinal dysbiosis with changes in the gut microbiota, rendering these patients prone to heart failure.

In our series, 94.44% of the patients developed diarrhea which LLF counteracted before the 5th day. LLF may exert is anti-inflammatory effect at gastrointestinal level, both balancing the local microbiota and reducing the intestinal damage induced by the virus. Lactoferrin increases the good micro flora—such as bifidus—and reduces bad bacteria, such as E. coli, streptococcus, clostridium and others.

Lactoferrin acts as an anti-inflammatory agent, promoting the “good” cytokines such as interleukin (IL)-4 and IL-10, and reducing the proinflammatory cytokines such as tumor necrosis factor-alpha, IL-6 and IL-1 beta, and downregulation of the nuclear factor-kappa. The best aspect of the treatment with LF is that it is simple, safe and potentially effective in the preventation and treatment of COVID 19 infections. It may be useful on young adults, elderly persons, children and women, regardless of their underlying pathology.

Additionally, we should highlight the mediation by lactoferrin of inflammation induced by the infection caused by COVID-19, which leads to the activation of macrophages and dendritic cells. Neutrophils degranulate in the place of the injury and release massive amounts of LF. In turn, lactoferrin can increase the low responses or modulate the ultra-aggressive cytokine activity. Both effects control inflammation and help tissular repair post-trauma.

Lactoferrin needs to be encapsulated in a PC liposome to protect it from pepsin hydrolysis and from the acid pH in the stomach, which allows the glycoprotein to completely pass through the intestines and be absorbed.

To complement the description and facilitate the understanding of the characteristics of the invention, for illustrative purposes this descriptive memory includes a set of images which represent the following:

FIG. 1:—These two images show how the formulation prevents the entry of coronavirus in the cell, and when there is infection.

FIG. 2—Distribution of particle size per number of particles (%) obtained through DLS studies for A) lactoferrin liposomes, B) sodium ascorbate liposomes, and C) zinc sulphate liposomes.

FIG. 3: Absence or presence of dry cough from patients before treatment (day 0) and after the treatment (48 hours and 5 days).

FIG. 4—Absence or presence of muscular pain from patients before treatment (day 0) and after the treatment (48 hours and 5 days).

FIG. 5—Absence or presence of tiredness from patients before treatment (day 0) and after the treatment (48 hours and 5 days).

FIG. 6—Absence or presence of headache from patients before treatment (day 0) and after the treatment (48 hours and 5 days).

FIG. 7—Absence or presence of taste from patients before treatment (day 0) and after the treatment (48 hours and 5 days).

FIG. 8—Absence or presence of smell from patients before treatment (day 0) and after the treatment (48 hours and 5 days).

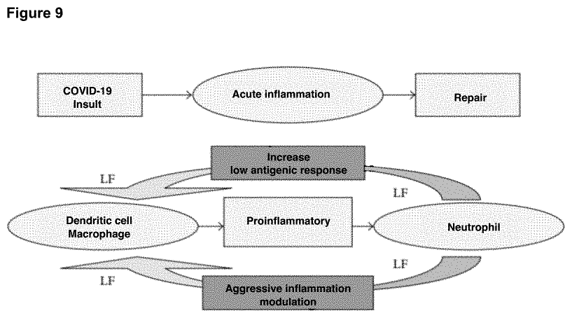

FIG. 9—Diagram representing the mediation by lactoferrin of inflammation induced by the infection caused by COVID-19, which leads to the activation of macrophages and dendritic cells.

### EXPLANATION OF EXECUTION MODE

Below is the description of the implementation method of the invention. On the one hand, it alludes to this invention, and on the other hand, to the use of said invention.

Soy lecithin for food purposes was purchased from Lipoid (Ludwigshafen, Germany); tween 20, from Comercial Quimica Massó (Barcelona, Spain); ethanol 96° from Panreac (Barcelona, Spain); lactoferrin from Ferrer Health (Barcelona, Spain); sodium ascorbate from Fagron Ibérica (Barcelona, Spain); and zinc sulphate heptahydrate from Nutrifoods (Barcelona, Spain).

The different liposomal solutions were prepared according to the procedure developed by the Sesderma laboratories (Valencia, Spain).(Serrano et al., 2015) Specifically, phospholipids (Phosphatidylcholine) were dissolved in ethanol 96° at room temperature, and then tween 20 was added to the ethanol solution. Simultaneously, the different active ingredients were dissolved in double-distilled water at a 5.2, 150 and 14 mg/ml concentration for lactoferrin, sodium ascorbate and zinc sulphate, respectively. Later, the lipidic mixture and the aqueous solution containing the corresponding active ingredient (lactoferrin, sodium ascorbate or zin sulphate) were combined and vigorously shaken for 5 minutes.

Finally, the resulting liposomal suspensions were filtered using a cellulose acetate syringe filter without surfactant, with a pore size of 0.2 μm.

The final concentration of the active ingredients in the different liposomal suspensions is detailed in the table below:

Active Ingredient Concentrations in the Different Liposomic Suspensions Prepared

| Active | Concentration |
|---|---|
| ingredient | (mg/ml) |
| lactoferrin | 4 |
| Sodiom | 100 |
| ascorbate | |
| Zinc | 10 |
| sulphate | |

The characterisation of the liposome in terms of particle size was evaluated through dynamic light dispersion (DLS). All measurements were carried out using a Delsa Nano C particle analyser (Beckman Coulter, Madrid, Spain), and performed three times in diluted liposome suspensions with filtered de-ionised water. Polydispersity index (PDI) was used as a measure to define homogeneity in size distribution. A sample presenting a PDI value equal to or lower than 0.2 is considered as a homogeneous population.

With the purpose of administering the encapsulated active ingredients to volunteers in the study, the prepared liposomal solutions were included in two liquid formulations of food supplement. The formulations of food supplements that consisted in a mixture of components with a nutritional a mixture of components with a nutritional physiological effect are shown in the table below:

### Composition of Food Supplements

| Food supplement 1 | Food supplement 2 |
|---|---|
| Citric acid | Citric acid |
| Potassium sorbate | Potassium sorbate |
| Sucralose | Sucralose |
| Aroma | Aroma |
| Sodium benzoate | Sodium benzoate |
| Xanthan gum | Xanthan gum |
| Glycerine | Glycerine |
| Sodium ascorbate liposomes | Zinc sulphate liposomes |
| Lactoferrin liposomes | |

The concentration of active ingredients in the different food supplement formulations is specified in the table below:

### Active Ingredient Concentration in the Prepared Formulation of the Food supplement (SA)

| | |
|---|---|
| Active | Concentration (mg/ml) |

| ingredient | SA1 | SA2 |
|---|---|---|
| Lactoferrin | 3, 2 | — |
| Sodium | 1, 2 | — |
| ascorbate | | |
| Zinc sulphate | — | 1, 1 |

Studies on particle size revealed that all the liposome formulations used in this study present a particle diameter in the interval of approximately 100-130 nm, with a PDI under 0.15. Results are shown in FIG. 2 and are summarised in the table below:

### Particle Diameter Measured by DLS and Polydispersity Index (PDI) of Lactoferrin Liposomes, Sodium Ascorbate and Zinc Sulphate

| Active | | |
|---|---|---|
| ingredient | | |
| encapsulated in | Particle | |
| liposomes | diameter (nm) | PDI |
| Lactoferrin | 124 ± 6 | 0.14 ± 0.03 |
| Sodium | 128 ± 8 | 0.06 ± 0.02 |
| ascorbate | | |
| Zinc sulphate | 130 ± 4 | 0.11 ± 0.03 |

Regarding the use of this composition, it must be done with the following doses of liposomal lactoferrin or amount:

Curative dose: 64-96 mg (20-30 ml) every 6h daily to cure the infection caused by COVID-19 (256-384 mg/day). Doses can be increased to 640-960 mg/day.

Preventive dose: one 64 mg dose, 2 or 3 times a day prevents the infection caused by COVID-19 (128-192 mg/day).

Pregnancy and babies (glycerosome encapsulation, alcohol free):
Pregnant women and babies under 2 years of age.
Mothers: 20 mg (6.25 ml) 3 or 4 times a day (72-80 mg/day).
Babies: 20 mg (6.25 ml) twice a day.
Zinc Defense syrup: 10-30 mg/day (10-30 ml)

In addition, regarding nasal administration of lactoferrin nasal drops (Lactyferrin™): these nasal drops contain nano-lactoferrin to quickly relieve acute sinusitis and smell and taste alterations experienced by many patients, while contributing to handling dry cough. In severe cases, we recommend applying 2 drops on each nostril every 2-3 hours for 12 hours, and then every 4-6 hours. Lactyferrin nasal drops are a very important factor for the relief of respiratory symptoms.

Once the nature of the invention has been adequately described, as well as an example of preferential implementation, it is clarified that the materials, shape, size and arrangements of the elements described can be modified, provided this does not entail an alteration of the essential characteristics of the invention, described below.

What is claimed is:

1. A composition to be used for treatment and prevention of infections caused by COVID-19 and other coronavirus, characterized by containing liposomal 5 lactoferrin as the main active ingredient.

2. A composition to be used for treatment and prevention of infections caused by COVID-19 and other coronavirus, according to claim 1, characterized by the fact that the concentration of liposomal lactoferrin in the formulation is between 2.9 mg/ml and 10 3.5 mg/ml, and preferably 3.2 mg/ml.

3. A composition to be used for treatment and prevention of infections caused by COVID-19 and other coronavirus, according to claim 1, characterized by the fact that the diameter of the particle of lactoferrin liposome is 124±6 nm.

4. A composition to be used for treatment and prevention of infections caused by COVID-19 and other coronavirus, according to claims 3, characterized by the fact that the phospholipid used to produce the lactoferrin liposomes is phosphatidylcholine.

5. Composition to be used for treatment and prevention of infections caused by COVID-19 and other coronavirus, according to claim 1, characterized by the fact that, besides the main active ingredients (lactoferrin), it contains liposome-encapsulated zinc salt in a concentration of between 1.0 mg/ml and 1.2 mg/ml, preferably 1.1 mg/ml.

6. A composition to be used for treatment and prevention of infections caused by COVID-19 and other coronavirus, according to claim 5, characterized by the fact that zinc salt is preferably zinc sulphate.

7. Composition to be used for treatment and prevention of infections caused by COVID-19 and other coronavirus, according to claim 6, characterized by the fact that, besides the main active ingredients (lactoferrin) and a secondary active ingredient (zinc salt liposomes), it contains vitamin C liposomes in a concentration of between 1.1 mg/ml and 1.3 mg/ml, preferably 1.2 mg/ml.

8. Use of the composition of previous claims for the treatment and prevention of infections caused by COVID-19 and other coronavirus using the following doses of liposomal lactoferrin:
curative dose: 64-96 mg (20-30 ml composition) every 6 h every day to cure the infection caused by COVID-19 (256-384 mg liposomal lactoferrin/day)
preventive dose: 64 mg (20 ml composition) 2 or 3 times a day (128-192 mg liposomal lactoferrin/day).

9. Use of the composition of previous claims for the treatment and prevention of infections caused by COVID-19 and other coronavirus using a dose of liposomal lactoferrin that can be increased to 960 mg/day.

10. Use of the composition of previous claims for the treatment and prevention of infections caused by COVID-19 and other coronavirus on pregnant women and babies under 2 years of age suing the following dose of liposomal lactoferrin:
mothers: 20 mg (6.25 ml) 3 or 4 times a day (72-80 mg/day).—babies: 20 mg (6.25 ml) 2 times a day.

11. A therapeutic method for treating infection of Corona Virus-19, comprising the steps of:
A) assessing and documenting predetermined symptoms for Corona Virus-19 infections; and
B) administering orally an effective amount of lactoferrin with a liposome carrier, over a predetermined period of time, to reduce said symptoms.

12. The method set forth in claim 11 further including an effective amount of vitamin C liposomes.

13. The method set forth in claim 12 further including an effective amount of zinc salt liposomes.

14. The method set forth in claim 13 wherein said effective amounts of lactoferrin, vitamin C and zinc salt liposomes are administered daily.

15. The method set forth in claim 14 wherein said effective amounts of lactoferrin is kept between 256 mg and 384 mg per day.

16. The method set forth in claim 15 wherein said effective amount of lactoferrin between 64 mg and 96 mg every 6 hours.

17. The method set forth in claim 16 wherein said symptoms are in the group that consists of tiredness or weakness, loss of smell, loss of taste, cough, muscular pain, headache and diarrhea.

18. A therapeutic method for preventing Corona Virus-19 infection, comprising the steps of:
A) assessing and documenting predetermined symptoms of a patient; and
B) administering orally an effective amount of lactoferrin in a liposome, over a predetermined period of time to prevent infection.

19. The method set forth in claim 18 further including an effective amount of vitamin C liposomes.

20. The method set forth in claim 19 further including an effective amount of zinc salt liposomes.

21. The method set forth in claim 20 wherein said effective amounts are administered daily.

22. The method set forth in claim 21 wherein said effective amounts of lactoferrin is kept between 128 mg and 192 mg per day.

23. The method set forth in claim 22 wherein said effective amount of lactoferrin is 64 mg between two and three times per day.

24. A therapeutic method for treating infection of Corona Virus-19, comprising the steps of:
A) assessing and documenting the symptoms of a patient; and
B) administering via nebulizer an effective amount of lactoferrin, over a predetermined period of time, to reduce said symptoms.

25. The method set forth in claim 24 wherein said effective amount is administered daily.

26. The method set forth in claim 25 wherein said effective amount of lactoferrin is not less than 64 mg and no more than 96 mg every six hours.

27. A therapeutic method for preventing Corona Virus-19 infection, comprising the steps of:
A) assessing and documenting that a patient lacks any symptoms; and
B) administering via nebulizer an effective amount of lactoferrin, over a predetermined period of time.

28. The method set forth in claim 27 wherein said effective amounts are administered daily.

29. The method set forth in claim 28 wherein said effective amount of lactoferrin between 128 mg and 192 mg per day.

30. The method set forth in claim 29 wherein said effective amount of lactoferrin is 64 mg given between two and three times per day.
